(19) United States
(12) Patent Application Publication
(10) Pub. No.: US 2012/0093791 A1
(43) Pub. Date: Apr. 19, 2012

(54) STIMULATION OF INNATE IMMUNITY WITH AN ANTIGEN FROM BACTERIAL ORIGIN

(76) Inventors: Cormier Yvon, Neuville (CA); Mélissa Girard, Quebec (CA); Evelyne Israël-Assayag, Quebec (CA)

(21) Appl. No.: 13/378,245

(22) PCT Filed: Jun. 15, 2010
(86) PCT No.: PCT/CA2010/000917

§ 371 Date: Dec. 14, 2011

Related U.S. Application Data
(60) Provisional application No. 61/187,040, filed on Jun. 15, 2009.

(51) Int. Cl. A61K 35/74; A61P 31/04; A61P 31/12; A61P 11/00

(52) U.S. Cl. 424/93.43; 424/93.4; 424/780

(57) ABSTRACT

There is provided a method of attenuating a respiratory infection in a subject, use of a composition for the manufacture of a medicament and a composition for treating respiratory infections comprising a preparation of Saccharopolyspora rectivirgula (SR), a non pathogenic thermophilic actinomycetes, which can stimulate the innate pulmonary immune response. This invention thus presents a novel preventive measure for acute viral or bacterial lung infections.

### RELATED APPLICATIONS

This application claims priority from U.S. provisional application 61/187,040 filed on Jun. 15, 2009 entitled “Stimulation of innate immunity with an antigen from bacterial origin” which is incorporated by reference in its entirety.

### FIELD OF THE INVENTION

The present invention relates to method and composition for increasing lung innate immunity against pathogens by administering a killed actinomycetes, crude extract or purified preparation thereof, a group of Gram-positive filaments forming bacteria, including Saccharopolyspora rectivigula (SR). Particularly, the purpose of this invention is to implement the concept that a bacterial lysate of Saccharopolyspora rectivirgula (SR), a non pathogenic thermophilic actinomycetes, and other actinomycetes, can stimulate the innate pulmonary immune response and thus presents a novel preventive measure for acute viral, bacterial or fungal lung infections.

### BACKGROUND OF THE INVENTION

According to the World Health Organization (WHO) acute respiratory infections continue to be the leading cause of acute illnesses worldwide. The populations most at risk for developing a fatal respiratory disease are the very young children, the elderly, and the immuno-compromised. The main etiological agents causing acute respiratory infections are viruses (eg. respiratory syncytial virus (RSV), human parainfluenza virus type 1, 2, and 3, influenza viruses) and several bacterial species (eg. Streptococcus pneumoniae, Haemophilus influenzae type b and, Staphylococcus aureus).

Influenza viruses alone globally infect 10 to 20% of the population every year causing up to 500,000 deaths. Influenza pandemics tend to recur on a fairly regular basis. Considering the insufficient supply of drugs or that the existing ones could be ineffective, the high specificity of vaccines for a particular virus strain, the limited vaccine production capacity, and a surge in demand for antiviral therapy with the progression of a pandemic, the development of preventive treatments is of the utmost priority. The recent 2009 outbreak of Influenza A H1N1 exemplifies the delays and difficulties of developing a timely and appropriate vaccine.

Due to continuous exposure to surrounding antigens, the lung is susceptible to infection by a large array of infectious agents. The annual worldwide mortality associated with lung viral and bacterial infections is important, and particularly in elderly and children. Between 4 000 and 8 000 Canadians die of influenza and its complications annually, depending on the severity of the season. Episodes of pandemic influenza have accounted for as many as 50 million deaths. H5N1 avian influenza has already caused more than 240 human deaths and a new pandemic influenza outbreak is predicted. Human to human transmission of this virus could cause even greater mortality than the Spanish flu of 1918. Also, as the respiratory system is an important portal of entry for pathogens, respiratory infectious agents could be used as bioterrorism and warfare agents.

Previous studies showed that local administration of bacteria, bacterial lysates or components can induce a protective response in the respiratory system against viruses, bacteria and fungus (1-6). For example, Clement et al. and Tuvim et al. reported that UV-killed non-typeable Haemophilus influenzae (NTHi) lysates protect mice against Streptococcus pneumoniae and Influenza A virus (H3N2) (4, 6). The optimal protective effect lasted for 24 h and waned rapidly thereafter. They also showed that for protection against Influenza A to be completely effective, the aerolized lysate must be given in multiple treatments or combined with ribavirin, an antiviral drug (6).

A review of prior art has highlighted ongoing research into bacterial products to prevent or attenuate respiratory infection. For example, U.S. Pat. No. 7,329,409 describes lysates of S. aureus, K pneumoniae or P. aeruginosa prepared with phages as immunomodulators for the treatment of microbial infections and for the enhancement of resistance to infection. U.S. Pat. No. 3,608,066 describes a preparation based on bacterial antigens (lyophilized killed streptococci, staphylococci, and pneumococci, Escherichia, Enterococci, Proteus, and Pseudomonas) admixed together with a propellant as a pharmaceutically suitable inhalation carrier for use in the immunotherapy of both the upper and lower parts of the respiratory tract. GB2021415 describes a medicament for treating infections of the respiratory passages that contains as the principal active substance a bacterial lysate obtained from a mixture of bacteria including S. aureus or S. viridians or N. catarrhalis and H. influenzae type b, or D. pneumoniae or K. pneumoniae or K. ozaenae or S. pyogenes. WO2006/084477 describes lyophilized bacterial extracts obtained by mechanical or alkaline lysis of bacteria (S. aureus, S. pyogenes, S. viridans, K. pneumoniae, K. ozaenae, H. influenzae serotype B, N. catarrhalis and D. pneumoniae) that can be effectively used for the preparation of a medicament for the prevention of tuberculosis relapse, to be administered in association. US2008/0170996 describes compositions, formulations and methods for the enhancement of a subject's biological defenses against infection, for example the subject's innate immunity against infection. Lysates described are composed of nontypable H. influenza (NTHi) or P. aeruginosa or E. coli or S. aureus or S. pneumonia. WO2008/085549 describes compositions and methods for stimulation on lung innate immunity. For example, they disclose using several bacterial strains for stimulating innate immunity but they do not disclose using Actinomycetes for this purpose.

Saccharopolyspora rectivirgula (SR) is a thermophilic actinomycete found in poorly conserved and mouldy hay, straw, or grain and is responsible for Farmer's lung (FL), one of the most common forms of hypersensitivity pneumonitis (HP) in North America. FL is a rare inflammatory lung disease (less than 3 per 1000 dairy farmers in Eastern Canada are affected by the disease) caused by an exacerbated immune response to repeated inhalations of large quantities of SR (7). The disease is characterized by a pulmonary infiltration and proliferation of activated lymphocytes. In the bronchoalveolar lavage fluid (BALF) of patients with HP, the absolute number and percentage of T cells are increased to as high as 80% of the recovered cells. However, at its early stages, the disease is fully curable just by removal from exposure to the causative agent. The mechanisms involved in the pathogenesis of HP are complex. There are increasing evidences that promoting factors are required for the onset of the disease. Few individuals exposed to an SR-contaminated environment develop clinical symptoms whereas more than 50% of Quebec dairy farmers develop a moderate lymphocytic alveolitis but remain asymptomatic with no evidence of lung damage when followed up for 20 years (8). These persons seem to develop a tolerant response to SR antigens.

None of the identified patents or patent applications describing the use of bacterial lysates or components as inducers of protection against respiratory pathogens teach the use of Saccharopolyspora rectivirgula (SR) or other actinomycetes.

There is a need for an alternative treatment to vaccines and antiviral drugs that offers non-selective protection against a broad spectrum of respiratory infections. Our findings represent such a treatment as our results indicate that topical administration of Saccharopolyspora rectivirgula (SR) lysate triggers a non selective protection in the lung against a respiratory virus.

### SUMMARY OF THE INVENTION

Here, we report that administration with an intranasally instilled lysate or extract of Saccharopolyspora rectivirgula (SR) or other actinomycetes protects mice against Sendai virus infection and other pulmonary infections. The current assumption is that our technology can replace vaccines as more rapid, easy to administer and readily available treatment regardless of the type or strain of a respiratory pathogen.

In a first aspect, the present invention therefore provides a killed preparation of an actinomycetes, a crude extract or a purified fraction thereof This preparation is useful for the stimulation of innate immunity and can therefore be used for the prevention or attenuation of a respiratory infection in a subject.

In a second aspect, the present invention provides composition comprising a killed preparation of an actinomycetes, a crude extract or a purified fraction thereof in admixture with a pharmaceutically acceptable excipient.

It is therefore a third aspect of the present invention to provide the use of a killed preparation of actinomycetes, a crude extract or a purified fraction thereof, or a composition as defined above in the manufacture of a composition for use in the prevention or attenuation of a respiratory infection in a subject.

It is also a fourth aspect of the present invention to provide a method for preventing or attenuating a respiratory infection in a subject, the method comprising administering a killed preparation of actinomycetes, a crude extract or a purified fraction thereof, in an amount sufficient to induce innate pulmonary immunity in the subject, thereby attenuating symptoms or severity of such infection.

### DETAILED DESCRIPTION OF THE INVENTION

### BRIEF DESCRIPTION OF THE DRAWINGS

Having thus generally described the aspects of the invention, reference will now be made to the accompanying drawings, showing by way of illustration, particular embodiments thereof, and in which:

FIG. 1 shows the survival of mice having been intranasally instilled for 3 consecutive days per week during 3 weeks with the SR antigenic lysate (200 μg in 50 μl), or Saline (control group). On week 3, a single Sendai virus inoculation of 25000 PFU was administered by intranasal instillation to a subgroup of SR or saline pre-treated animals. Control groups included SR and saline treated animals non-infected with Sendai. Ten days after the virus instillation, only 20% of mice survived to the viral infection whereas none of the SR+Sendai group died from the viral infection.

FIG. 2 shows the SR protective effect on mice infected with the Sendai virus. Groups of mice were anesthetized with isoflurane and treated by the intranasal route for 3 consecutive days per week during 3 weeks with the SR antigenic lysate (200 μg in 50 μl), or Saline (control group). On week 3, a single Sendai virus inoculation at a sublethal dose of 5000 PFU was administered by intranasal instillation to a subgroup of SR or saline pre-treated animals. Control groups included SR and saline treated animals non-infected with Sendai. Clinical signs were noted and the animals were sacrificed 6 days or 9 days after viral infection, this timing correlates with the peaks of virus multiplication and lung damage respectively. Bronchoalveolar lavages (BAL) were performed and the number of cells in the BAL fluid counted as a measure of cellular inflammation. On days 6 and 9 after the virus inoculation, SR-treated animals did not show any clinical signs whereas untreated mice showed clear clinical signs of weight loss and shivering. On day 9 post-Sendai, cellular inflammation in SR-pretreated mice infected with the virus returned to normal values compared to Sendai infected untreated animals. These results indicate that multiple SR treatments clearly protected against Sendai virus infection.

FIG. 3 shows a histology staining of lung tissue from mice intranasally instilled with: saline (3 days per week for 3 weeks), Sendai virus (5 000 PFU; only one instillation on week 3), SR (200 μg/50 μl), 3 days per week for 3 weeks) or SR+Sendai. Sacrifices were performed 9 days post-Sendai. Inflammatory parameters were scored on an arbitrary scale. Mice infected with Sendai virus without SR pre-treatment showed a peribronchiolar and perivascular hyperplasia and more severe pulmonary tissue damages than mice from SR and SR+Sendai groups. FIG. 3a shows the lung histology staining from one animal from each group. FIG. 3b shows the mean histology scores for all mice from each group

FIG. 4 shows the lung viral load in mice intranasally instilled with: saline (on day 1, 2, and 3), Sendai virus (5 000 PFU; only one instillation on day 3), SR (200 μg/50 μl), on days 1, 2, and 3) or SR+Sendai. Sacrifices were performed on day 12 (9 days post-Sendai). Lungs were homogenized and lysis plaques were counted on Hep-2 cells. Lung tissue from mice of the Sendai group demonstrated an increased number of viral particles compared to the SR+Sendai group.

FIG. 5 shows a time-related anti-viral protection of mice having received a single nasal instillation of: saline, SR (200 μg), Sendai (5000 PFU) or SR+Sendai (indicated hours are times of Sendai virus administration after SR instillations). Mice were sacrificed on day 9 post-Sendai. A marked attenuation of the virus-induced inflammation was observed when SR was given 24 hours prior to the Sendai virus administration and no clinical signs were observed in SR pre-treated mice compared to untreated. These results demonstrate the efficacy of a single SR treatment in preventing virus-induced inflammation and that this protective effect is present up to 5 days after a single SR treatment.

FIG. 6 shows a long term duration of SR-induced viral protection in mice administered with a single nasal instillation of saline, SR (200 μg), Sendai (5000 PFU) or SR+Sendai (indicated hours are times of Sendai virus administration after SR instillations). Mice were sacrificed on day 9 post-Sendai. An attenuated virus-induced inflammation is observed if SR is given 5 or 7 days prior to the Sendai virus administration. No protection is observed if SR is given more than two weeks after the Sendai infection indicating that the duration of the protection is up to at least 7 days but less than 14 days after a single treatment with SR. These results indicate that the preventive SR treatment can be administered from 24 hours to up to 7 days prior to the viral infection.

FIG. 7 is a western blot protein profile for the total lysate of SR, the supernatant, or the pellet.

FIG. 8 shows that treatment with SR supernatant (SRs) 72 hours before the Sendai virus protects against subsequent viral infections as efficiently as the whole SR lysate. Inflammatory cells in bronchoalveolar lavage (BAL) from the SR group had returned to normal values. Cell counts in SR lysate or SR lysate supernatant (SRs) pre-treated animals infected with the virus (SR+Sendai, SRs+Sendai groups) had also returned to normal and the animals showed no adverse clinical signs. Animals infected with Sendai virus alone had persistent inflammatory cells. These results demonstrate that the supernatant fraction of the SR lysate is as active as the total lysate in attenuating the virus-induced lung inflammation and symptoms.

FIG. 9 shows that exposure to SR lysate supernatant confers a protection against respiratory syncytial virus.

FIG. 10 shows that exposure to SR lysate supernatant confers a protection against mouse adenovirus-1.

FIG. 11 shows that exposure to SR lysate supernatant confers a protection against Influenza A H1N1 virus.

FIG. 12 shows that exposure to SR lysate supernatant confers partial protection against Pseudomonas aeruginosa.

FIG. 13 demonstrate that mice from the SRs+P. aeruginosa group show a decreased percentage of epithelial cells in the bronchoalveolar lavage fluids (4.57%) as compared to mice exposed to P. aeruginosa only (10.75%).

FIG. 14 illustrates that lungs from mice infected with P. aeruginosa without SR lysate supernatant pre-treatment showed a higher colony forming units (CFU) number in the lung homogenates (5.7×10⁵ CFU) compared to mice from the SRs+P. aeruginosa group (0.37×10⁵ CFU).

FIG. 15 shows that SRs treatment prior to S. pneumoniae infection induces no apparent protective effect. S. pneumonia alone did not induce a significant cellular inflammation, therefore no statistical difference was observed between cells counts from S. pneumoniae (0.083×10⁶ cells/ml) and SRs+S. pneumoniae (0.115×10⁶ cells/ml) groups of mice. Number of colony forming units (CFU) in mice lung homogenates might be affected and needs to be evaluated before concluding that SRs pretreatment is not protective against S. pneumoniae.

FIG. 16 shows that other actinomycetes protect against a Sendai virus infection as well as does SR.

FIG. 17 shows the long-term safety of SR lysate supernatant exposure in mice intranasally instilled with: saline (a single treatment per week for 12 weeks), or SR lysate supernatant (a single treatment per week for 12 weeks). Sacrifices were performed at different time points (24 h, 72 h, 7 days, or 14 days) after the last instillation. These results demonstrate that the cellular influx caused by a single intranasal instillation per week for 12 weeks of the SR lysate supernatant has almost returned to normal values 14 days after the last antigen instillation and suggest that long-term exposure to SR lysate supernatant causes a mild inflammation that wanes rapidly. Most importantly, the number of inflammatory cells from BAL fluids of mice that received a single instillation of SR lysate supernatant per week for 12 weeks are much lower compared the BAL cell number in mice that received 3 instillations of SR lysate supernatant per week for 3 weeks (FIG. 2). This suggests that one weekly long-term exposure to SR lysate supernatant is safe.

FIG. 18 shows that the neutrophilic influx caused by a long-term exposure to SR lysate supernatant (a single instillation per week for 12 weeks) has completely disappeared 14 days after the last SR lysate supernatant instillation.

FIG. 19 shows stained lung tissue sections from mice having been intranasally instilled with saline (a single administration per week for 12 weeks) or SR lysate supernatant (a single administration per week for 12 weeks). Sacrifices were performed 24 hours and 7 days after the last SR administration. Results show that cellular infiltration caused by long-term exposure to SR lysate supernatant rapidly decreases 7 days after the last SR administration (FIG. 19c) compared to 24 h post SR (FIG. 19b) and appears similar to a normal lung section (FIG. 19a).

FIG. 20 shows the long-term protective effect of SR lysate supernatant exposure in mice having been intranasally instilled with: saline (a single administration per week for 12 weeks), SR lysate supernatant (a single administration per week for 12 weeks), Sendai virus (3000 PFU; a single instillation on week 13), or SR lysate supernatant plus a single instillation of Sendai virus 72 hours after the last SR lysate supernatant instillation. Sacrifices were performed 9 days post-virus infection. Results show that long-term exposure to SR lysate supernatant (once a week for 12 weeks) does not affect the protective effect. Mice that received SR lysate supernatant administration prior to Sendai virus infection show a marked decrease of inflammatory cells in BAL fluids compared to mice that only received a Sendai virus instillation.

FIG. 21 shows that long-term exposure to SR lysate supernatant (a single instillation per week for 12 weeks) protects against the weight loss caused by a Sendai virus infection.

### DETAILED DESCRIPTION OF PARTICULAR EMBODIMENTS

In a first aspect, the present invention provides a killed preparation, a crude extract or a purified fraction of an actinomycetes.

In a second aspect, the present invention provides the use of a composition comprising a killed preparation, a crude extract or a purified fraction of actinomycetes, in the manufacture of a composition for the prevention or attenuation of a respiratory infection in a subject.

In a third aspect, the present invention provides a composition comprising a killed preparation, a crude extract or a purified fraction of an actinomycetes, in admixture with a physiologically acceptable excipient, for use in the prevention or attenuation of symptoms of a respiratory viral infection in a subject.

In a fourth aspect, the invention provides a method of preventing or attenuating a respiratory infection in a subject, the method comprising administering a killed preparation of an actinomycetes, a crude extract or a purified fraction thereof, in an amount sufficient to induce innate pulmonary immunity in the subject and thereby attenuate symptoms or severity of such infection.

### Bacterial Preparation and Lysis

Particularly, the bacterial preparation can be killed by irradiation (such as, for example, UV irradiation) or by lysis. Particularly, the crude extract of an actinomycetes is produced by lysing a solution or suspension of bacteria by conventional means such as: repeated freeze-thaw cycles, sonication, homogenization with beads, use of chemicals, detergents or enzymes. Therefore, particularly, the method comprises the administration of this lysate. In particular instances, the lysate comprises bacterial components such as: DNA, RNA, proteins, peptides, lypopolysaccharides, organelles and cell wall membrane components. Particularly, the lysate can be centrifuged and the pellet can be separated from the supernatant fraction, each fraction being suitable for administration. More particularly, the supernatant fraction is used for making the composition of the invention.

Still more particularly, the purified fractions of the bacterial extract can be achieved by the addition of proteases, protein separation by sucrose gradient, solvent extraction of lipids, extraction of RNA/DNA by using columns with silica-membrane technology, chromatography system separations.

### Actinomycetes

The composition as defined above comprises a preparation of actinomycetes selected from the group consisting of: Nocardioforms (nocardia, rhodococcus, nocardioides, pseudonocardia, oerkscovia, saccharopolyspora, faenia, promicromonospora, Intrasporangium, actinopolyspora and saccharomonospora); multilocular sporangium (geodermatophilus, dermatophilus, frankia); Actinoplanetes (actinoplanes, ampullariella, pilimelia, dactylosporangium; micromonospora); Streptomycetes (Streptomyces, streptoverticillium, kineosporia, sporichthya); Maduromycetes (actinomadura, microbispora, microtetraspora, planobispora, planomonospora, spirillospora, streptosporangium); Thermonospora (thermonospora, actinosynnema, noardiopsis, streptoalloteichus); Thermoactinomycetes (thermoactinomyces); glycomyces; kibdelosporangium; kitasatosporia; saccharothrix; and pasteuria.

More particularly, actinomycetes are selected from the group consisting of: Saccharopolyspora rectivigula (SR), Streptomyces sp., Saccharomonospora viridis (SV), Thermoactinomyces vulgaris (TV), and Saccharopolyspora hirsutis (SH).

Still more particularly, the killed actinomycetes is a non-pathogenic actinomycetes. Alternatively, the actinomycetes is a thermophilic actinomycetes.

### Subject

Particularly, the subject being treated is a mammal or a bird susceptible to respiratory infections, such as for example, human, horse, cat, dog, pig, and birds (such as for example, migratory birds, fowl or poultry). More particularly, the subject is a human who is at risk of developing or has developed such a respiratory infection.

### Mode of Administration

Particularly, the invention comprises administering the preparation to the respiratory tract such as locally or topically. More particularly via nasal or oral (buccal) route. More particularly, the composition may be administered by inhalation, nebulization, nasal spray, aerosol, instillation, in the form of a liquid or powder.

Particularly, the invention comprises the manufacture of a composition being formulated for delivery to the respiratory tract such as for local or topical administration. More particularly the composition is formulated for nasal or oral (buccal) administration. More particularly, the composition may be formulated for inhalation, nebulization, nasal spray, aerosol or instillation.

Particularly, the composition is formulated for delivery to the respiratory tract, particularly for local or topical administration. More particularly the composition is formulated for nasal or oral (buccal) administration. More particularly, the composition may be formulated for inhalation, nebulization, nasal spray, aerosol or instillation.

Particularly, the composition is formulated for delivery to the respiratory tract in combination with other bacterial lysates or other anti-viral and anti-bacterial agents (e.g. antibiotics).

Still, particularly, the composition is formulated for delivery to the respiratory tract in a range of dosings sufficient to induce an innate immune response.

### Frequency of Administration

The method also comprises administering topically a single dose or multiple doses of killed preparation of actinomycetes, a crude extract or a purified fraction thereof at least 24 h to up to 7 days or more.

### Respiratory Infections

Particularly, the invention of the invention is useful for the prevention or attenuation of all respiratory viruses including, but not restricted to Picornaviridae, Adenoviridae, Paramyxoviridae, Orthomixoviridae, Coronaviridae, Filoviridae, Pneumoviridae, Retroviridae, Poxviridae, Herpesviridae and then more specific genius rhinovirus, mastadenovirus, aviadenovirus, paramyxovirus, pneumovirus, influenzavirus (type A and B), human respiratory syncytial virus, vaccinia virus, coronavirus, and Ebola.

More particularly, the invention is indicated for the prevention or attenuation of all respiratory viruses including, but not restricted to, Picornaviridae (rhinovirus), Adenoviridae (mastadenovirus, aviadenovirus), Paramyxoviridae (respiratory syncytial virus (RSV), parainfluenza viruses type 1, 2, and 3), Orthomyxoviridae (influenza virus), Coronaviridae (coronavirus), Filoviridae, (Ebola virus). Still, more particularly, the method of the invention is useful for the prevention or attenuation of rhinovirus, respiratory syncytial virus (RSV), parainfluenza viruses type 1, 2, and 3, and influenza virus type A and B. Most particularly, the method is useful for the prevention or attenuation of Sendai virus (parainfluenza virus).

Particularly, the invention is useful for the prevention or attenuation of all respiratory infections caused by bacteria including, but not restricted to, Gram-negative and Gram-positive bacteria such as for example, Streptococcus pneumoniae, Haemophilus influenzae type b, Bacillus anthracis and Staphylococcus aureus.

More particularly, the invention is useful for the prevention or attenuation of respiratory infections caused by a pathogen such as Sendai virus, respiratory syncytial virus (RSV), adenovirus, influenza virus such as for example, H1N1 and H5N1, Pseudomonas aeruginosa, or Streptococcus pneumoniae.

Also particularly, the invention is useful for the prevention or attenuation of respiratory fungal infections for example Aspergillus fumigatus, Candida, Pneumocystis.

Recently, we discovered that mice intranasally treated with a preparation of SR lysate and subsequently infected with a lethal dose of Sendai (parainfluenza) virus, develop an effective immune defense mechanism against the virus. A single administration of SR lysate gave a protection against Sendai virus that lasts for up to 7 days. At the same viral dose, mice infected with the virus alone developed a clinically obvious respiratory infection, often leading to death of the animals. Mice that had received a single nasal administration of SR, 7 days prior to the virus instillation had no clinical signs of infection, minimal lung inflammation, and decreased virus loads in the lungs.

A mouse model was developed where groups of mice were exposed to the SR lysate for 3 weeks, then infected with a sublethal dose of Sendai virus and re-exposed to SR for 9 more weeks (SR+virus). Control groups included mice exposed to SR lysate alone and mice non-exposed to SR lysate and infected with the virus alone. In the group of mice infected with an overwhelming dose of virus without SR pre-treatment, 10 out of 12 mice died within days. The two surviving mice in the virus-infected group were obviously ill and had a large neutrophilic inflammation in the lung. No mortality was observed in the group of mice exposed to SR lysate, and subsequently infected with the Sendai virus. We hypothesized that SR lysate could activate a pulmonary immune response, mostly that of the innate immune system due the rapid and non specific response, and offer a protection from a subsequent viral infection. As SR lysate induces a non-specific protection in the respiratory system, we coined this technology Respiratory Innate Immune System Activator (RIISA).

This method provides a novel way of protecting humans and other mammals from viral and bacterial infections by priming our own defense system. Compared to vaccines which only protect against one specific virus and one strain of that viral family, our technology has a broader protective activity against several viruses and also bacteria. The bacterial lysate could be given by inhalation (e.g. puffer) during local outbreaks in endemic regions, or worldwide in pandemics of influenza virus outbreaks. Another potential application is the protection against biothreat agents. Indeed, SR lysate could be readily available and easy to administer in all kinds of field situation.

Existing vaccination methods are dependent on stimulating the body's immune system against one or several specific strains, but since influenza viruses mutate continually, existing vaccines are likely to be totally ineffective against new emerging strains. Moreover, when a new pandemic strain emerges, it takes several months to produce a new strain-specific vaccine for worldwide vaccination, administer it to susceptible populations, and for the recipient to develop specific immunity.

The technology we are proposing could offer a (i) broader protection against any pathogenic strains and/or agents, (ii) complete protection within hours of the administration, (iii) a protection that lasts up to 7 days and which could potentially be maintained indefinitely by a weekly administration. Since the lysate could potentially offer protection for various strains of virus/agents, it could be mass produced in advance, stockpiled and be available immediately when needed, iv) the product being given by inhalation could be self-administered therefore making its large scale distribution very cost effective (no need for the mobilization of nurses and doctors to administer the composition).

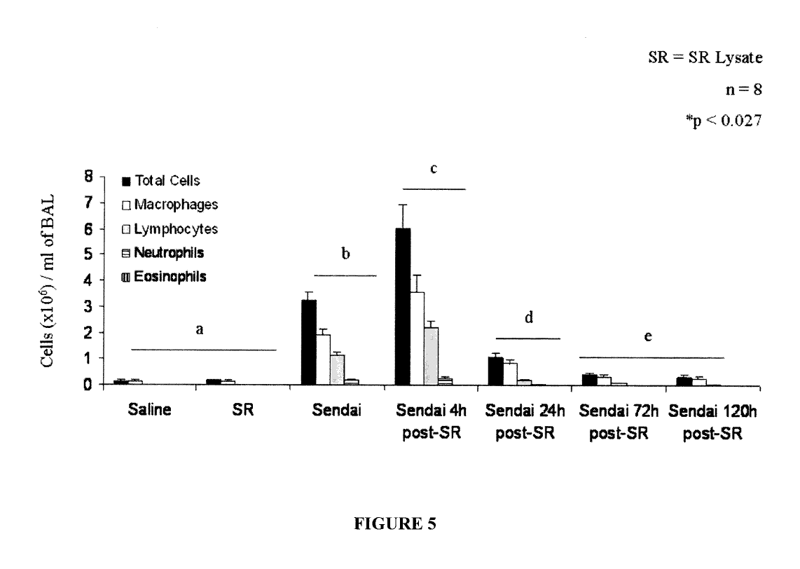

Previous studies by Clement et al. (4) have described the use of bacterial lysates to stimulate the innate immunity in the lungs. However, their bacterial lysate provides protection for up to 24 hours and daily inhalation is required for long term protection, and thus this would not only be impractical, but also lead to ongoing lung inflammation with a potential for eventual parenchymal lung damage. For this reason, as described in their US2008/0170996, these inventors had to limit the repeated administration of their antigen to a maximal of ten. The novel SR lysate described here, administered once a week allows sufficient time for all the induced inflammatory response to wane before the subsequent administration (FIG. 5). The bacterial preparation that we propose is produced from non pathogenic thermoactinophilic bacteria with an optimal growth at 50° C. The production of the lysate also kills all viable bacteria. Moreover, we have shown that farmers exposed daily to SR often develop an immune response to this antigen but remain free of any pulmonary alterations after a 20 years follow-up (8).

Some reported studies address the mechanism of the protective effect induced by bacterial components (4,6) These studies showed that the non specific protection is provided by activation of the innate immunity in the respiratory system, the best protection provided by local administration of bacterial product. Clement et al. reported an increase in multiple antimicrobial polypeptides in the lung lining fluid such as lysozyme, lactoferrin, haptoglobin, calgranulin, and surfactant apoprotein D expressed by epithelial cells and leucocytes as well as other mediators such as interferons (4). They also showed that protection against lethality does not depend on neutrophils recruitment to the lungs but can be associated with interferon signaling (4, 6).

The mechanisms involved in the immunoregulatory event induced by SR lysate are yet to be determined. However based on available literature and without wishing to be bound by theory, there are several theoretical pathways via which the protection could be achieved. Innate immunity is conferred by many antimicrobial molecules such as the defensins and collectins in addition to those described by Clement et al. Cell surface, intracellular and cytosolic pattern recognition receptors (PRR) including Toll-like receptors (TLR) are also implicated in bacterial sensing. More than one PRR and thus more than one components of SR are probably involved in the detection and the triggering of the innate immune system (9). For example, TLR2 which mediates host response to Gram positive bacteria such as SR (10), and TLR9 which recognizes unmethylated CpG in bacterial genome, could potentially be involved in the induced protective effect against Sendai virus infection. In addition to the sensors of bacterial components, some PRR are involved in virus sensing such as TLR7, and the triggering of these PRR by infecting virus will add to the global innate immune response in the lung in SR treated and virus infected mice (11).

### EXAMPLES

### Example 1

### Repeated SR Exposure Induced Viral Protection

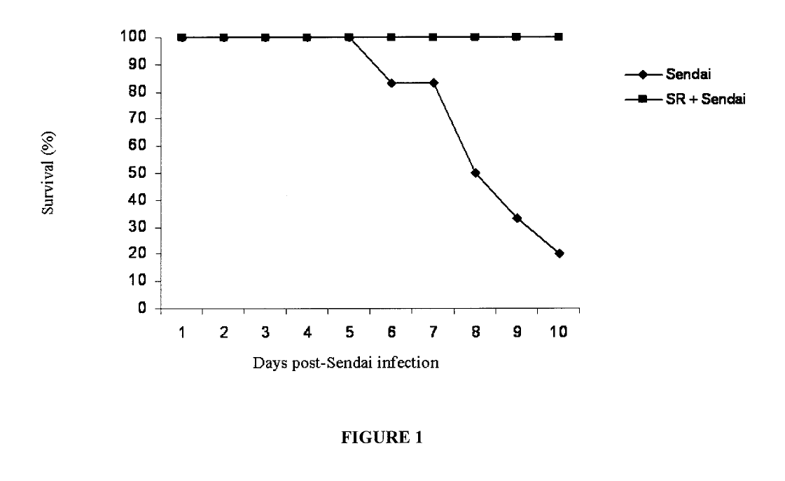

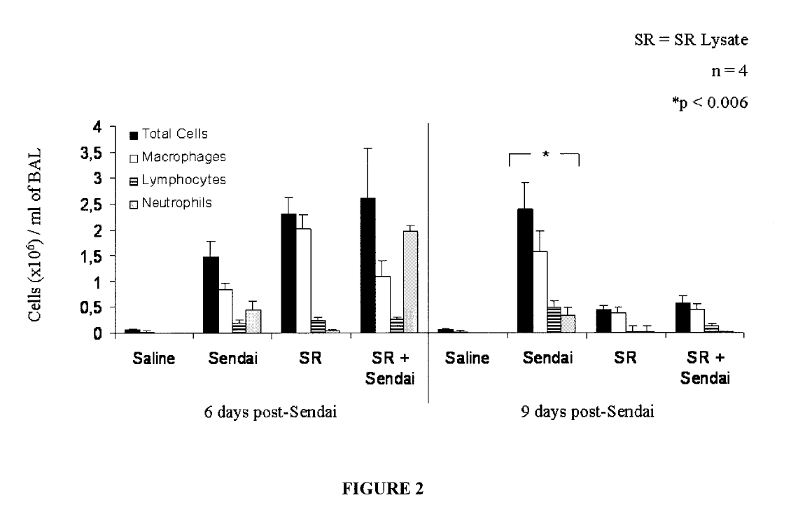

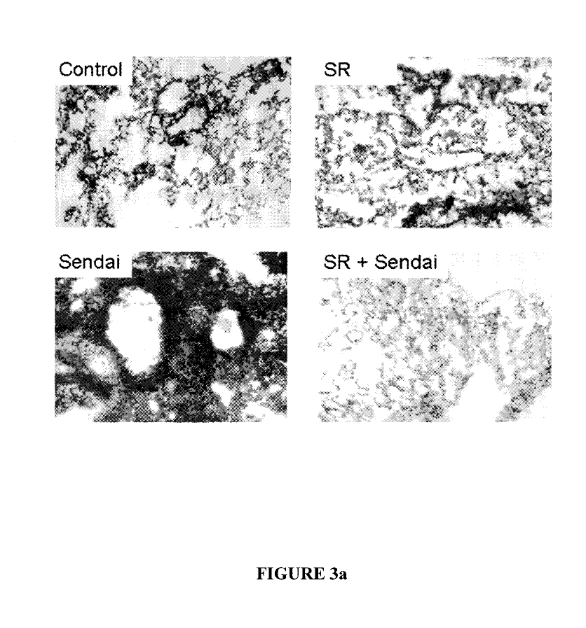

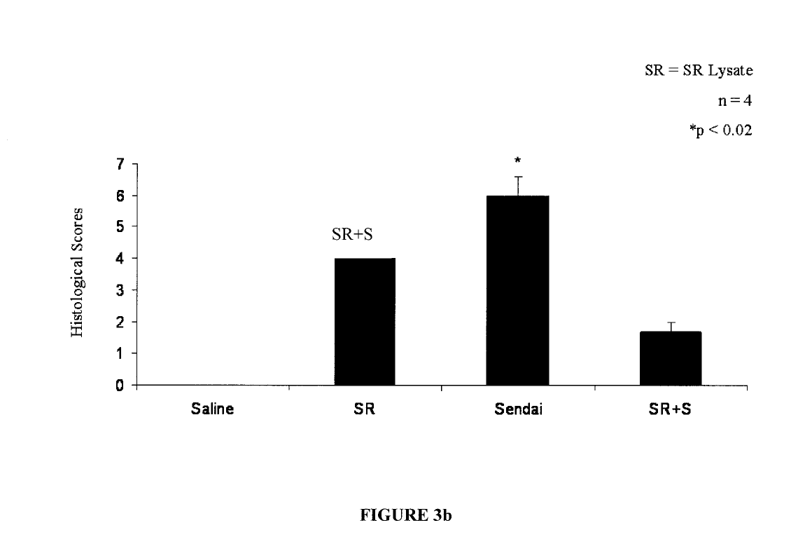

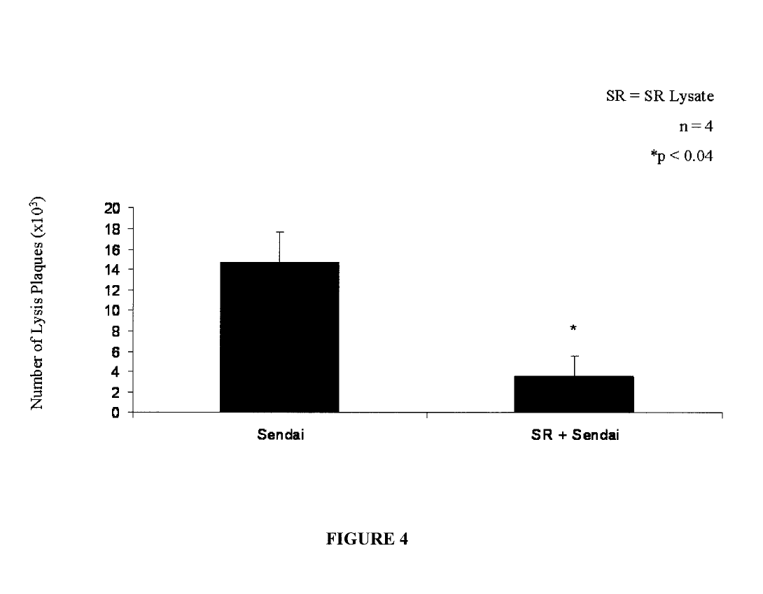

Our hypothesis of the SR-induced innate immune response stimulation stemmed from the observation that animals infected with a high dose of Sendai virus (25000 PFU) had 20% survival rate whereas animals infected with the same dose but pre-treated with SR had 100% survival rate (FIG. 1). Subsequently, a sublethal dose (5000 PFU) of Sendai virus was used for inoculation and the extent of lung inflammation determined as a marker of SR-treatment efficacy. The animals were exposed to the SR lysate for 3 days a week for 3 weeks prior to the Sendai virus infection, no further SR exposure after the viral infection was performed. The mice were sacrificed 6 or 9 days post-Sendai infection which are respectively the peak for virus multiplication and the peak for pulmonary damages. Inflammatory cell counts in bronchoalveolar lavage (BAL) showed that at 9 days post-Sendai infection, the cell counts for mice from the SR group have returned to normal values since the SR-specific inflammatory response decreases as soon as the antigenic exposure is stopped (FIG. 5). Cell counts from animals exposed first to SR and then infected with the virus (SR+Sendai group) had also returned to normal values. Animals that were infected with Sendai virus alone had a very high number of inflammatory cells in the BAL (FIG. 2), lost more weight, had more severe pulmonary tissue damages and inflammatory cell infiltration (FIGS. 3a and 3b), and had a higher virus titer in lung homogenates (FIG. 4) compared to the high inflammatory response in mice from the SR+Sendai group. These results demonstrate that exposure to the SR lysate 3 days a week for 3 weeks protected mice from Sendai virus infection by controlling the viral replication and the pulmonary inflammatory response.

### Example 2

### A Single SR Administration Induces Viral Protection

In another set of experiments we verified whether (i) one single dose of SR lysate could be sufficient to confer protection and (ii) the duration of the protective effect. Groups of mice received intranasally (i.n.) SR lysate 4 h, 24 h, 72 h and 120 h before infection with Sendai virus. Other groups included mice pre-treated with SR lysate without infection and mice infected without pre-treatment, as well as control untreated non-infected mice. BAL analyses were performed on day 9 post-Sendai infection. The results clearly indicate that one unique SR lysate treatment administered between 72 and 120 hours before the viral infection is sufficient to confer a protection against the Sendai virus infection (FIG. 5).

### Example 3

### Long Term Duration of SR-Induced Viral Protection

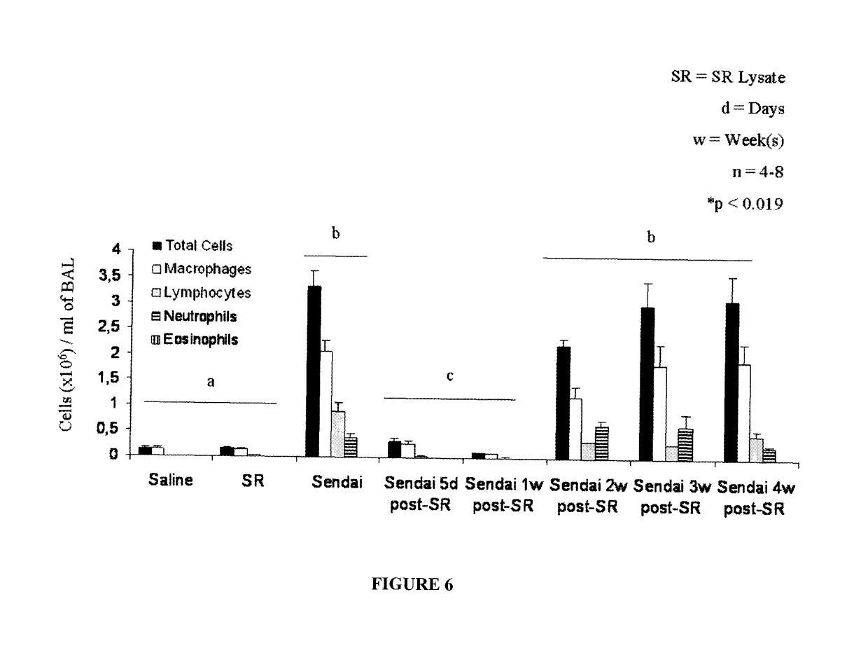

The results from the previous study indicated that one single intranasal exposure to the SR lysate induced a protective effect up to 5 days after the viral infection. The next study was aimed to determine the maximal duration of the protective effect of SR exposure. The study protocol was designed to assess the duration of the effect for up to 8 weeks after the SR treatment. Groups of mice were administered by intranasal route the SR lysate 5 days, 1 week, 2 weeks, 3 weeks, 4 weeks and 8 weeks before being infected with Sendai virus. Control groups were administered intranasal saline alone, SR lysate treatment without infection, and Sendai virus without SR lysate treatment. BAL analyses were performed on day 9 post Sendai virus infection. Results presented in FIG. 6 indicate that intranasal SR lysate induces an efficient protection against Sendai virus infection up to 7 days after treatment.

### Example 4

### Proteome Identification of Saccharopolyspora rectivirgula

### Electrophoretic Analysis

SDS-PAGE analysis was carried out using BioRad Criterion XT Bis-Tris precast gel (4-12% acrylamide) according to the manufacturers's protocol. Protein samples of Saccharopolyspora rectivirgula from the total lysate, supernatant, and pellet have been dissolved in sample preparation buffer, heated and then loaded onto the gel. Following electrophoresis, proteins have been visualized by Sypro ruby staining.

### LC-MS/MS Analysis

A SDS-PAGE protein lane was cut into 32 gel slices per lane using a disposable lane picker (The Gel Company, CA, USA). Gel slices were deposited into 96-well plates. In-gel protein digest was performed on a MassPrep™ liquid handling station (Waters, Mississauga, Canada) according to the manufacturer's specifications and using sequencing-grade modified trypsin (Promega, Madison, Wis., USA). Peptide extracts were dried out using a SpeedVac™.

Peptide extracts were separated by online reversed-phase (RP) nanoscale capillary LC (nanoLC) and analyzed by electrospray MS (ES MS/MS). The experiments were performed on a Thermo Surveyor MS pump connected to a LTQ linear ion trap mass spectrometer (Thermo Electron, San Jose, Calif., USA) equipped with a nanoelectrospray ion source (Thermo Electron, San Jose, Calif., USA). Peptide separation was done within a PicoFrit column BioBasic C18, 10 cm×0.075 mm internal diameter (New Objective, Woburn, Mass., USA) with a linear gradient from 2% to 50% solvent B (acetonitrile, 0.1% formic acid) in 30 min, at 200 nl/min. Mass spectra was acquired using data-dependent acquisition mode (Xcalibure software, version 2.0). Each full-scan mass spectrum (400-2000 m/z) was followed by collision-induced dissociation of the seven most intense ions. The dynamic exclusion function was enabled (30 s exclusion), and the relative collisional fragmentation energy was set to 35%.

### Interpretation of Tandem MS Spectra

All MS/MS samples were analyzed using Mascot (Matrix Science, London, UK; version 2.2.0). Mascot was set up to search against Saccharopolyspora erythrae protein database (genome is already sequenced) assuming a digestion with trypsin. Fragment and parent ion mass tolerance were, respectively, of 0.5 Da and 2.0 Da. Iodoacetamide derivative of cysteine was specified as a fixed modification. Oxidation of methionine was specified as variable modifications. Two missed cleavages were allowed.

### Criteria for Protein Identification

Scaffold (version 02_{—}05_{—}00; Proteome Software Inc., Portland, Oreg., USA) was used to validate MS/MS-based peptide and protein identifications. Peptide identifications were accepted if they could be established at >80.0% probability as specified by the Peptide Prophet algorithm. Protein identifications are accepted if they could be established at >90.0% probability and contained at least two identified peptides. Protein probabilities were assigned by the Protein Prophet algorithm. Proteins that contained similar peptides and could not be differentiated based on MS/MS analysis alone were grouped to satisfy the principles of parsimony. Using these stringent identification parameters, the rate of false positive identifications was <1%.

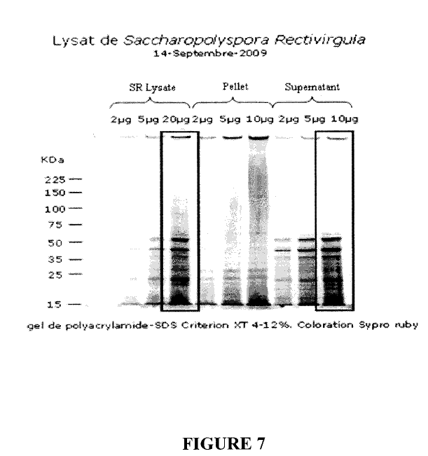

FIG. 7 shows the protein profile for the total lysate of SR, the supernatant, or the pellet. The supernatant and the total lysate show a similar protein profile. Determination of SR proteins led to the identification of 1248 proteins.

### Example 5

### Protective Effect of SR Lysate Supernatant

The SR lysate preparation was spun down, the supernatant collected and tested for its potential protective properties on Sendai-infected mice. Groups of mice were slightly anesthetized with isoflurane and intranasally treated with either saline, the whole SR lysate (200 μg in 50μl) or the soluble fraction of the SR lysate (50 μl). The pre-treated animals were subsequently infected with a single dose of Sendai virus (5 000 PFU) by intranasal route 72 h later. A control group of untreated, uninfected mice was included. The number of inflammatory cells in the bronchoalveolar lavage was evaluated 9 days after Sendai infection. This timing correlates with the peak of Sendai-induced lung inflammation.

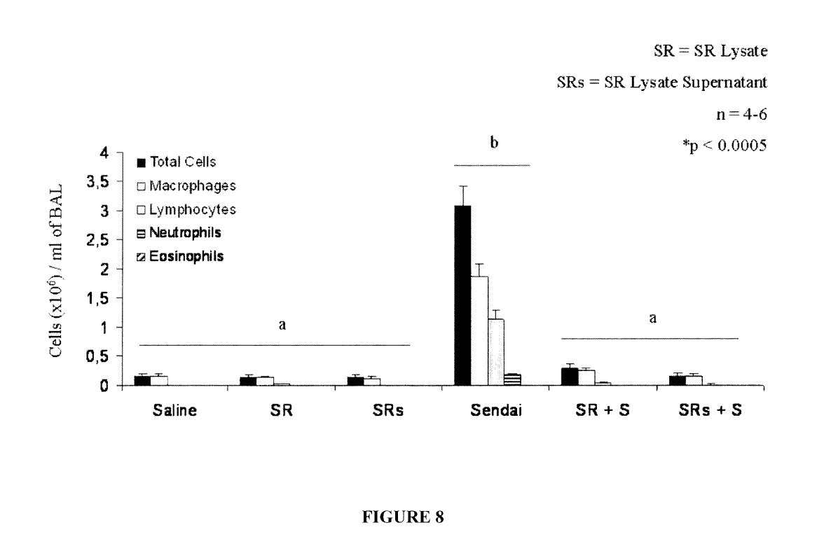

The results of FIG. 8 indicate that treatment with SR supernatant (SRs) 72 hours before Sendai virus infection protects against a subsequent viral infection as efficiently as the whole SR lysate. Inflammatory cells in bronchoalveolar lavage (BAL) from the SR group returned to normal values. Cell counts in SR lysate or SR lysate supernatant (SRs) pre-treated animals infected with the virus (SR+Sendai, SRs+Sendai groups) indicate minimal inflammation. Animals infected with Sendai virus alone had marked inflammatory cells (3.071×10⁶ cells/ml for Sendai vs 0.295×10⁶ cells/ml for SR+Sendai and 0.157×10⁶ cells/ml for SRs+Sendai). These results demonstrate that the supernatant fraction of the SR lysate comprising the protein profile identified in FIG. 7 as well as other unidentified components is as active as the total lysate for protecting against viral challenge.

### Example 6

### SR Lysate Supernatant-Induced Protective Effect Against other Pathogens

The protective effect of SR lysate supernatant was evaluated against 3 other strains of viruses and 2 strains of bacteria. Groups of mice were pre-treated with the SR-lysate supernatant and subsequently infected 72 hours later with either respiratory syncytial virus (RSV) (10⁵ TCID₅₀), mouse adenovirus (MAV-1) (5×10⁴ TCID₅₀), influenza virus H1N1 (1.87×10³ TCID₅₀), Pseudomonas aeruginosa (3×10⁷ CFU), or Streptococcus pneumoniae (10⁷ CFU). Bronchoalveolar lavages analyses were performed 7 days after the infection and inflammatory cells were counted as a marker of lung inflammation. In the P. aeruginosa infected groups of either untreated or SR-treated mice, the number of epithelial cells was also counted and the number of colony forming unit (CFU) in the lung homogenate determined.

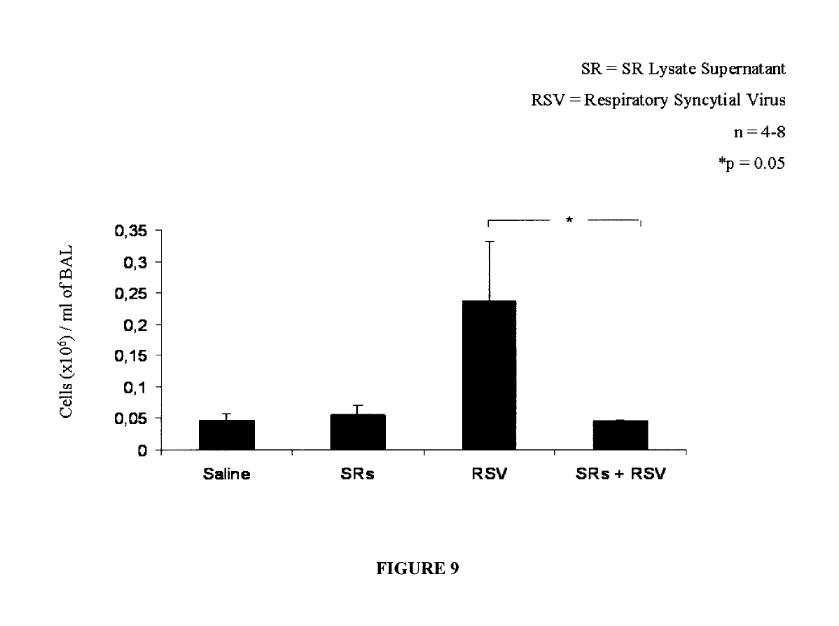

FIG. 9 shows that exposure to SR lysate supernatant confers a protection against respiratory syncytial virus. Total cells counts from mice treated with SR lysate supernatant before RSV infection (SRs+RSV group) are markedly lower than those in mice that received the RSV virus alone (0.046×10⁶ cells/ml for SRs+RSV vs 0.184×10⁶ cells/ml for RSV).

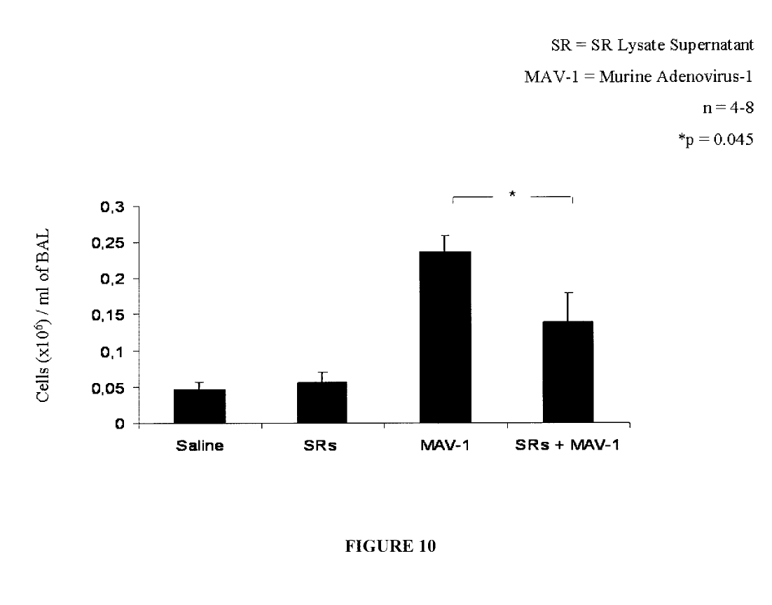

FIG. 10 shows that exposure to SR lysate supernatant confers a protection against mouse adenovirus-1. Animals pre-treated with SRs and then infected with the MAV-1 (SRs+MAV-1 group) show a significant decrease in total cell counts compared to mice from the MAV-1 group (0.183×10⁶ cells/ml for SRs+MAV-1 vs 0.237×10⁶ cells/ml for MAV-1).

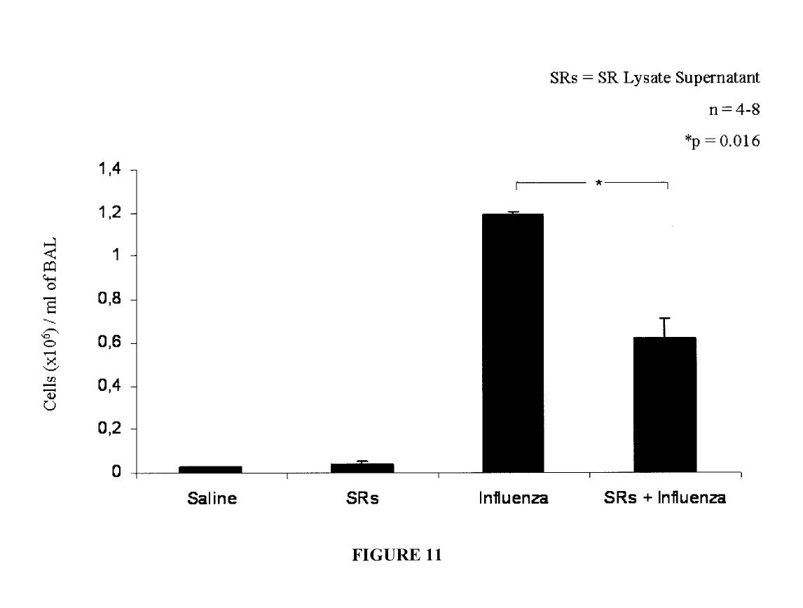

FIG. 11 shows that exposure to SR lysate supernatant confers a protection against Influenza A/PR/34/8 (H1N1) virus. A decrease in total cell counts in mice treated with SR lysate supernatant before Influenza inoculation (SRs+Influenza group) is observed compared to the number of cells recovered from mice that had received the Influenza virus only.

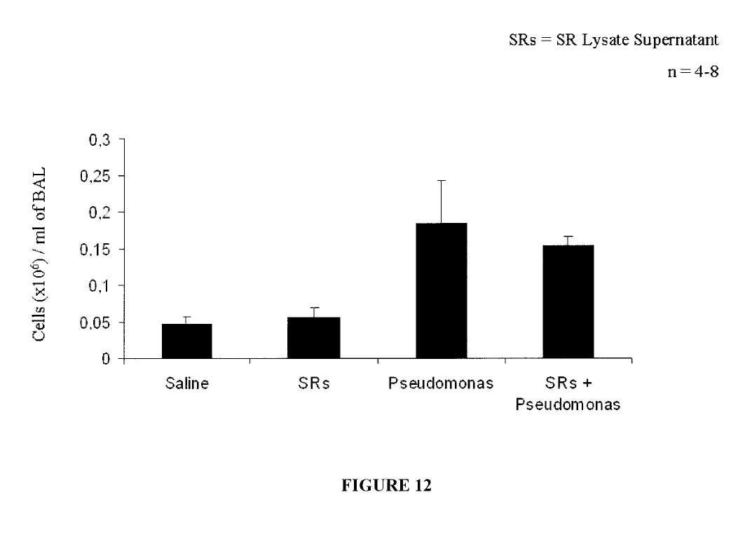

FIG. 12 shows that exposure to SR lysate supernatant confers partial protection against Pseudomonas aeruginosa. Mice from the SRs+P. aeruginosa group show a slight decrease in the total cell counts (0.154×10⁶ cells/ml) compared to P. aeruginosa group of mice (0.185×10⁶ cells/ml).

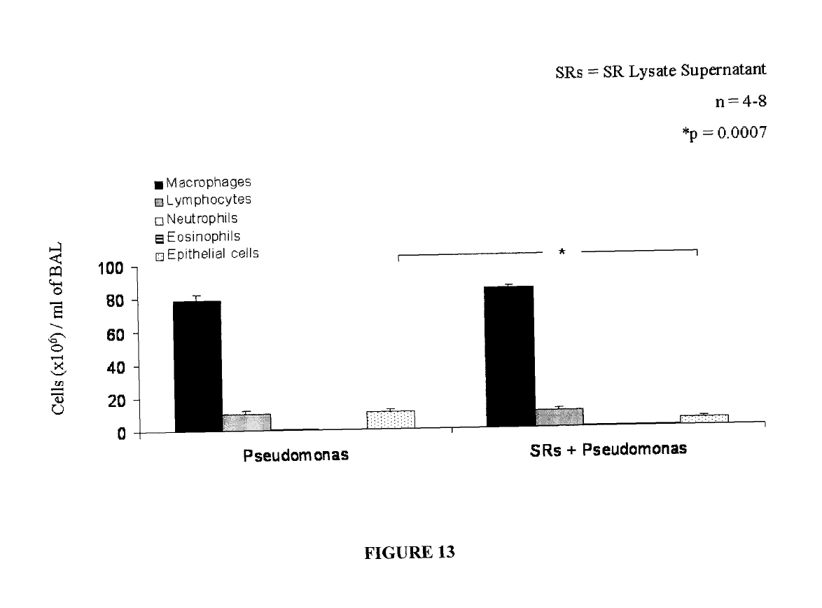

FIG. 13 demonstrate that mice from the SRs+P. aeruginosa group show a decreased percentage of epithelial cells in the bronchoalveolar lavage fluids (4.57%) as compared to mice exposed to P. aeruginosa only (10.75%).

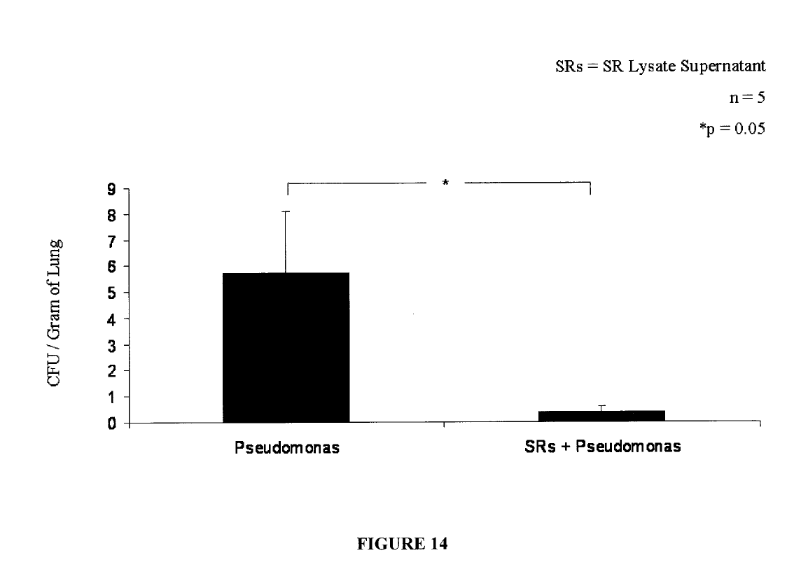

FIG. 14 illustrates that lungs from mice infected with P. aeruginosa without SR lysate supernatant pre-treatment showed a higher colony forming units (CFU) number in the lung homogenates (5.7×10⁵ CFU) compared to mice from the SRs+P. aeruginosa group (0.37×10⁵ CFU). Administration of SR lysate supernatant prior to a P. aeruginosa infection protects against epithelial desquamation and bacterial growth.

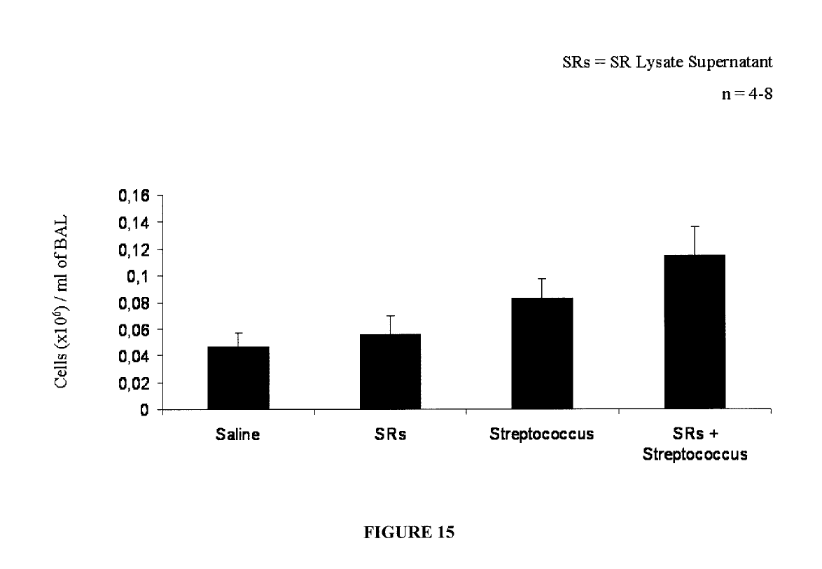

FIG. 15 shows that SRs treatment prior to S. pneumoniae infection induces no apparent effect on lung cellular inflammation since S. pneumonia alone did not induce a significant cellular inflammation, therefore no statistical difference was observed between cells counts from S. pneumoniae (0.083×10⁶ cells/ml) and SRs+S. pneumoniae (0.115×10⁶ cells/ml) groups of mice. A change in colony-forming unit (CFU) may however take place.

### Example 7

### Induction of Antiviral Protection by Other Actinomycetes

The potential antiviral protective effect of Saccharomonospora viridis (SV), Thermoactinomyces vulgaris (TV), and Saccharopolyspora hirsute (SH) was also evaluated. These actinomycetes were cultured for four days in Nutrient broth (SV at 45° C. and TV at 50° C.) or in yeast malt extract broth (SH at 37° C.), washed three times with sterile water, homogenized and lyophilized. The antigenic preparations were reconstituted with pyrogen-free saline at a concentration of 4 mg/ml, centrifuged at high speed and the supernatant used to pre-treat the mice 72 hours prior to the Sendai infection. The number of inflammatory cells in the bronchoalveoar lavage was counted on day 9 after the viral infection as a marker of lung inflammation.

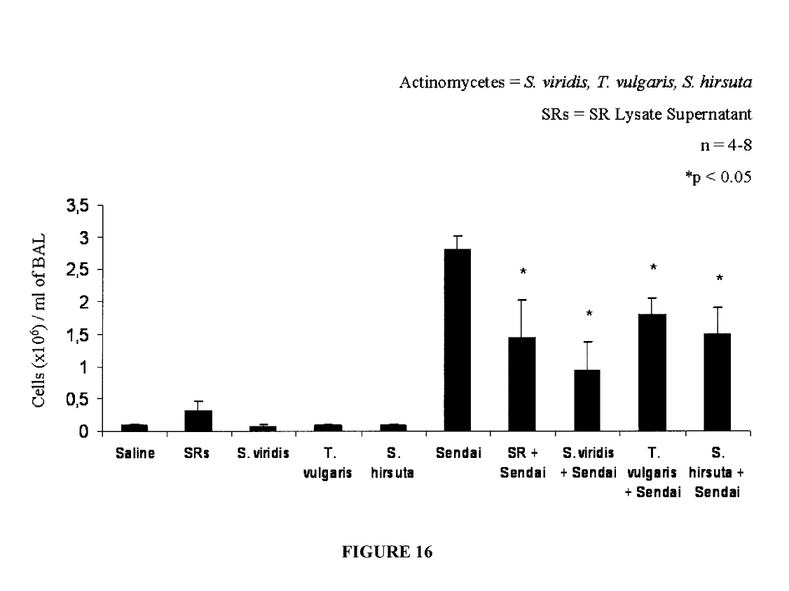

FIG. 16 shows the protective activity of other actinomycetes against Sendai virus infection. Inflammatory cells in bronchoalveolar lavage (BAL) of mice pre-treated with the various actinomycete lysate supernatants and subsequently infected with the virus (SRs+Sendai, SVs+Sendai, TVs+Sendai, and SHs+Sendai groups) were significantly decreased compared to untreated Sendai infected mice. These results indicate that similarly to the protection conferred by SR, other actinomycetes also efficiently protect against the cellular lung inflammation caused by a Sendai virus infection.

### Example 8

### Safety and Efficacy of Repeated Administrations of SR Lysate

The effects of long term, once a week SR treatments, on lung inflammation and histopathology were evaluated. The animals were intranasally instilled with SR lysate supernatant (200 μg) once a week for 12 weeks. A control untreated group was included. Inflammatory cell influx was measured and potential tissue lung damage was assessed 24 h, 72 h, 7 days, or 14 days later.

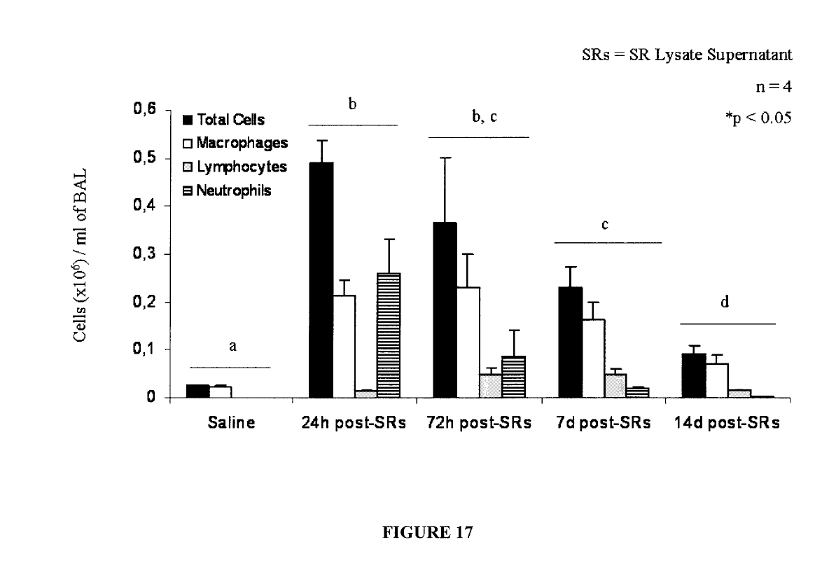

FIG. 17 shows the cellular influx caused by a single intranasal instillation per week of the SR lysate supernatant for 12 weeks in mice. Lung cellular inflammation and tissue damage were assessed 24 h, 72 h, 7 days, or 14 days after the last exposure. A small increase in total cell number is observed but much lower than the one observed after multiple SR administrations (FIG. 2). This number declines rapidly after cessation of SR exposure and has nearly returned to normal values 14 days after the last antigen instillation. These results suggest that long-term exposure to SR lysate supernatant causes a very low grade inflammation which disappears rapidly thereafter.

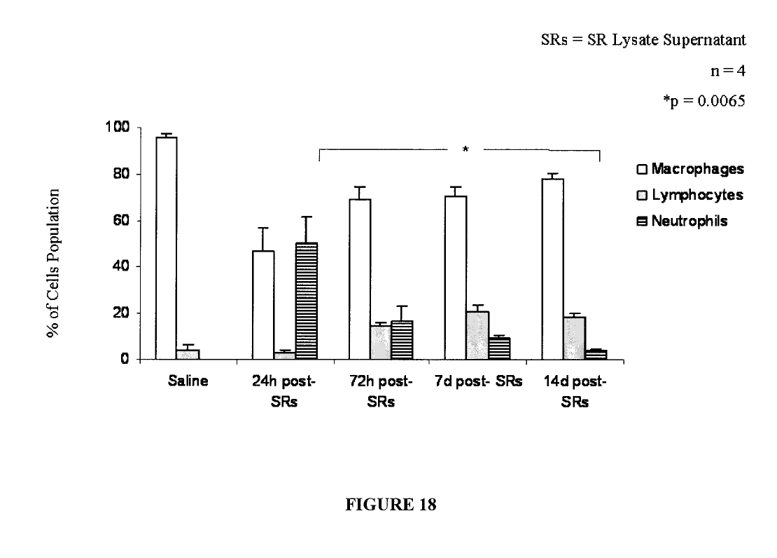

FIG. 18 shows that the neutrophilic influx caused by a long-term exposure to SR lysate supernatant (a single instillation per week for 12 weeks) has completely disappeared 14 days after the last SR lysate supernatant instillation.

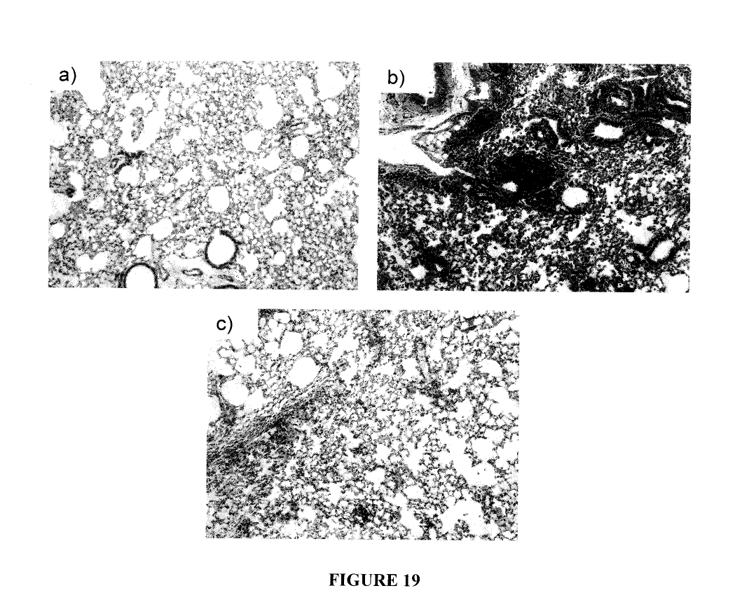

FIG. 19 shows a histopathology study of mice having been intranasally instilled with: a) saline (a single instillation per weeks for 12 weeks), or SR lysate supernatant (a single instillation per weeks for 12 weeks). Sacrifices were performed at 24 hours and 7 days after the last SR administration. Results show that cellular infiltration caused by long-term exposure to SR lysate supernatant rapidly decreases 7 days after the last administration (FIG. 19c) compared to 24 h post SR (FIG. 19b) and appears similar to a normal lung section (FIG. 19a).

The efficacy of long term administration on the protective effect was also evaluated. SR-treated animals were infected with Sendai virus, whereas control groups included (i) SR-treated, non-infected animals, (ii) untreated animals, infected with the Sendai virus, (iii) untreated, uninfected mice. Cellular inflammation and lung histopathology were determined 9 days after the viral infection.

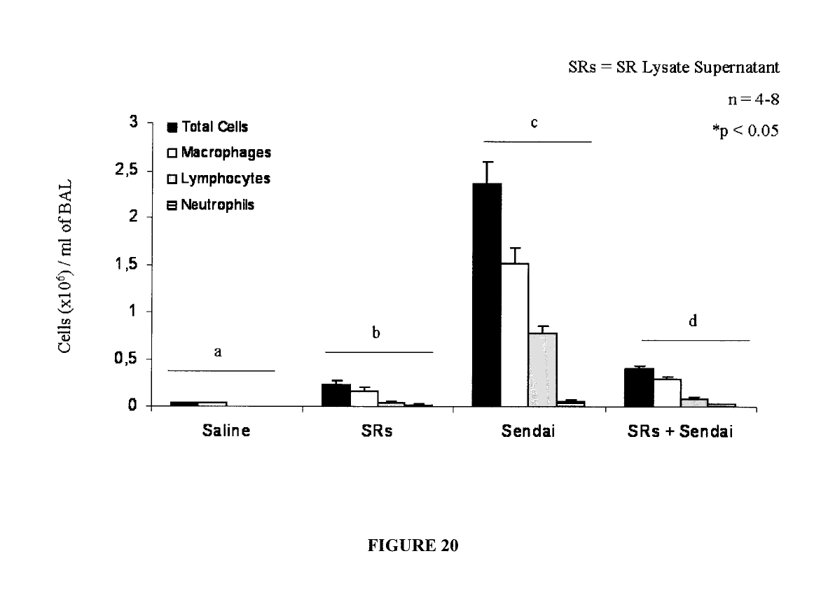

FIG. 20 shows the long-term protective effect of SR lysate supernatant exposure in mice intranasally instilled with: saline (a single instillation per weeks for 12 weeks), SR lysate supernatant (a single instillation per weeks for 12 weeks), Sendai virus (3000 PFU; a single instillation on week 13), or SR lysate supernatant plus a single instillation of Sendai virus 72 hours after the last SR lysate supernatant instillation. Sacrifices were performed 9 days post-virus infection. Results show that long-term exposure to SR lysate supernatant (once a week for 12 weeks) does not affect the protective effect. A marked decrease of inflammatory cells in the BAL of mice that received SR lysate supernatant administration prior to Sendai virus infection is observed compared to the cellular inflammation in untreated mice inoculated with the virus.

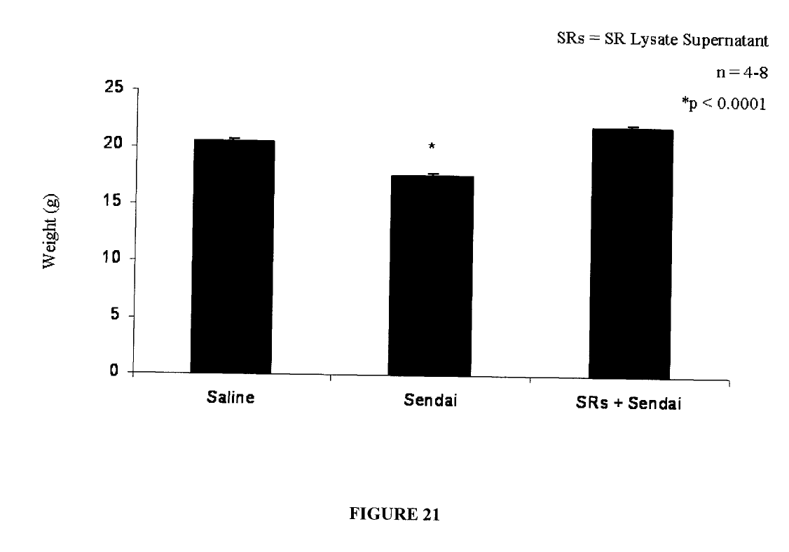

FIG. 21 shows that long-term exposure to SR lysate supernatant (a single instillation per weeks for 12 weeks) protects against weight loss caused by a Sendai virus infection. Mice were intranasally instilled with: saline (a single instillation per weeks for 12 weeks), SR lysate supernatant (a single instillation per weeks for 12 weeks), Sendai virus (3000 PFU; a single instillation on week 13), or SR lysate supernatant plus a single instillation of Sendai virus 72 hours after the last SR lysate supernatant instillation. Sacrifices were performed 9 days post-virus infection. Mice that did not received SR lysate supernatant administration prior to Sendai virus infection showed a marked weight loss (17.62 g) compared to the animals pre-treated with the SR lysate supernatant (21.98 g).

### Conclusion

These examples support our hypothesis that intranasal pre-treatment with a crude or supernatant of SR lysate induces a non specific protection against subsequent viral and bacterial infections.

Thus, our method offers obvious advantages compared to the technology developed by Clement et al.: (i) SR is a non pathogenic bacteria, (ii) the inflammatory neutrophilic response induced by a single exposure to SR is minimal and wanes rapidly (see FIG. 5), (iii) except for dairy farmers, the majority of the population is not in daily contact with SR thus is not expected to develop a potential allergenic sensitization, (iv) the protection induced intranasal administration of SR lysate lasts for several days, is efficient against a virus infection without antiviral agent, and (v) since a single weekly exposure is sufficient to induce protection, it is not damageable for the pulmonary system. Moreover, the efficacy is maintained after 12 weeks of a single lysate administration. This protection is also reproduced with the use of killed or crude preparation from other actinomycetes and is useful against various respiratory infections caused by a variety of pathogens.

### REFERENCES

1. Hori et al. Clin Diagn Lab Immuno. 2001 May; 8(3):593-7
2. Williams et al. J Immunol. 2004 Dec. 15; 173(12):7435-43,
3. Wong et al. Vaccine. 2005 Mar. 18; 23(17-18):2266-8.
4. Clement C G et al. Am J Respir Crit Care Med. 2008; 177(12): 1322-30.
5. Wong et al. doi:10.1016/j.vaccine.2009.01.048,
6. Tuvim M J et al. PLoS ONE 2009; 4 (1).
7. Cormier Y et al. Am Rev Respir Dis 1984; 130: 1046-1049.
8. Cormier Y et al. Eur Respir J 2004; 23: 523-25.
9. Ishi et al. Cell Host Microbe. 2008 Jun. 12; 3(6):352-63
10. Hallman Metal. Pediatr Res. 2001; 50(3): 315-21.
11. Iwamura et al. Curr Allergy Asthma Rep. 2008 March; 8(1):7-13

1. A composition comprising a killed suspension of an actinomycetes, a crude extract or a purified fraction thereof, in admixture with a pharmaceutically acceptable excipient.

2. The composition of claim 1, wherein said actinomycetes is selected from the group consisting of: Nocardioforms; multilocular sporangium; Actinoplanetes; Streptomycetes; Maduromycetes; Thermonospora; Thermoactinomycetes; glycomyces; kibdelosporangium; kitasatosporia; saccharothrix; and pasteuria.

3. The composition of claim 2, wherein said actinomycetes is selected from the group consisting of: nocardia, rhodococcus, nocardioides, pseudonocardia, oerkscovia, saccharopolyspora, faenia, promicromonospora, Intrasporangium, actinopolyspora, saccharomonospora, geodermatophilus, dermatophilus, frankia, actinoplanes, ampullariella, pilimelia, dactylosporangium, micromonospora, Streptomyces, streptoverticillium, kineosporia, sporichthya, actinomadura, microbispora, microtetraspora, planobispora, planomonospora, spirillospora, streptosporangium, thermonospora, actinosynnema, noardiopsis, streptoalloteichus, and thermoactinomyces.

4. The composition of claim 1, wherein said actinomycetes is selected from: Saccharopolyspora rectivigula (SR), Streptomyces sp., Saccharomonospora viridis (SV), Thermoactinomyces vulgaris (TV), and Saccharopolyspora hirsutis (SH).

5. The composition of claim 1, wherein said actinomycetes is anon-pathogenic thermophilic actinomycetes.

6. The composition of claim 5, wherein said actinomycetes is Saccharopolyspora rectivirgula.

7. The composition of claim 1, wherein said crude extract is prepared by lysis of a bacterial suspension of said actinomycetes.

8. The composition of claim 7, wherein said crude extract is a supernatant fraction from a lysed bacterial suspension of said actinomycetes.

9.-19. (canceled)

20. A composition as defined according to claim 1, being formulated for delivery to the respiratory tract for local or topical administration.

21.-22. (canceled)

23. A method of attenuating a respiratory infection in a subject, the method comprising administering a composition as defined in claim 1, in an amount sufficient to induce innate pulmonary immunity in the subject, thereby attenuating symptoms or severity of such infection.

24. The method according to claim 23, wherein said composition is administered via nasal or oral (buccal) route.

25. The method according to claim 24, wherein said composition is administered by inhalation, nebulization, nasal spray, aerosol or instillation.

26. The method according to claim 25, wherein said composition is administered in the form of a liquid or a powder.

27. A method for the preparation of a composition as defined in any one of claim 1, comprising the steps of:
a) killing a suspension or solution of an actinomycetes by irradiation or by lysis;
b) recovering a crude extract of said killed actinomycetes;
c) optionally centrifuging said crude extract to produce a supernatant fraction and a pellet fraction; and
d) optionally recovering said supernatant fraction.

28. The method according to claim 26, wherein said solution or suspension of actinomycetes is lysed in step a) by: repeated freeze-thaw cycles, sonication, homogenization with beads, use of chemicals, detergents or enzymes.

29. The method according to claim 23, wherein said composition is administered in combination with another treatment selected from the group consisting of: a bacterial lysate, an antiviral agent, and an anti-bacterial agent.
